(19) United States
(12) Patent Application Publication
(10) Pub. No.: US 2015/0225315 A1
(43) Pub. Date: Aug. 13, 2015

(54) REACTOR DESIGN FOR LIQUID PHASE FLUORINATION

(71) Applicant: HONEYWELL INTERNATIONAL INC., MORRISTOWN, NJ (US)

(72) Inventors: Haluk KOPKALLI, Staten Island, NY (US); Yuon CHIU, Denville, NJ (US); Daniel C. MERKEL, Orchard Park, NY (US); Ron Joseph ROOF, Center Valley, PA (US); Robert A. SMITH, Kinnelon, NJ (US); Austin V. PRANKE, Succasunna, NJ (US)

(73) Assignee: HONEYWELL INTERNATIONAL INC., MORRISTOWN, NJ (US)

(21) Appl. No.: 14/618,450

(22) Filed: Feb. 10, 2015

Related U.S. Application Data
(60) Provisional application No. 61/937,825, filed on Feb. 10, 2014.

(51) Int. Cl. C07C 17/087; B01J 19/24; B01J 19/18; C07C 17/25

(52) U.S. Cl. CPC C07C 17/087; C07C 17/25; B01J 19/248; B01J 19/18; B01J 2219/24; B01J 2219/00049

(57) ABSTRACT

A reactor apparatus is provided having a reaction chamber; a compartmentalizing apparatus including a plurality of compartments, each compartment being open at a top end and bottom end, the compartmentalizing apparatus being disposed within the reaction chamber; and an inlet disposed at an area of the reactor and in fluid communication between the reaction chamber and a feed source. Also provided is a reactor apparatus having an assembly having a reaction chamber; at least one agitator assembly configured to generate mixing within the reaction chamber, the agitator assembly including: a shaft partially lined or coated with a fluoropolymer or other non-metallic corrosion-resistant material, and an impeller lined or coated with the fluoropolymer or other non-metallic corrosion-resistant material; and an inlet disposed at an area of the reactor and in fluid communication between the reaction chamber and a feed source. The provided reactor apparatus can be utilized in hydrofluorination processes.

### I. RELATED APPLICATION

The present application claims priority of U.S. Ser. No. 61/937,825, filed on Feb. 10, 2014, the contents of which are incorporated by reference.

### II. FIELD OF THE INVENTION

The present invention relates to an apparatus useful for fluorinating organic compounds, or more particularly to a reactor suitable for the fluorination of organic compounds on a commercial scale.

### III. BACKGROUND OF THE DISCLOSURE

Fluorocarbons, particularly fluorinated olefins, as a class, have many and varied uses, including as chemical intermediates and monomers. In particular, these products are useful as refrigerants, monomers or intermediates for preparing refrigerants, particularly those identified as having low global warming potential.

With concerns over global warming, hydrofluoroolefins (HFOs) are being commercialized as substitutes for chlorofluorocarbons (CFCs), hydrochlorofluorocarbons (HCFCs) and hydrofluorocarbons (HFCs) for use as refrigerants, heat transfer agents, blowing agents, monomers and propellants because HFOs do not deplete the ozone layer and have low global warming potential. Some HFOs are prepared by multiple steps that involve fluorinating a chlorinated organic compound with a fluorination agent such as hydrogen fluoride in the presence of a fluorination catalyst. These reactions may be conducted in either the liquid or gas phase or a combination of these. In one process to manufacture 2,3,3,3-tetrafluoro-1-propene (HFO-1234yf), the following reaction sequence is preferred:

CCl₂═CClCH₂Cl+3HF→CH₂═CClCF₃+3HCl; Step 1:

CH₂═CClCF₃+HF→CH₃CClFCF₃; and Step 2:

CH₃CClFCF₃→CH₂═CFCF₃+HCl. Step 3:

In a preferred embodiment, Step 1 takes place in the gas phase in the presence of a fluorination catalyst, Step 2 takes place in the liquid phase in the presence of a fluorination catalyst and Step 3 takes place in the gas phase in the presence or absence of a dehydrochlorination catalyst.

For Step 2 of the above process, liquid phase fluorination is preferred because the reaction is controlled at relatively lower temperatures which results in less by-product formation due to oligomerization, decomposition, or overfluorination.

Liquid phase fluorination, however, uses and generates corrosive compounds, such as hydrogen fluoride, hydrogen chloride, and Lewis acid catalysts, which form superacids. These superacids tend to corrode the reactor vessel in which the reaction is conducted, even reactors comprised of corrosion-resistant materials such as Inconel 600, NAR25-50MII, Hastelloy C, Hastelloy G-30, duplex stainless steel, and Hastelloy C-22. Corrosion of the reactor compromises the structural integrity of the reactor and reduces its useful life. Therefore, a need exists to minimize reactor corrosion.

In liquid phase reactions, especially when the reaction components are immiscible or partially miscible, location of reactant introduction and mixing of the reaction mass is a very important criterion. In such reactions, the degree of mixing affects conversion, yield and selectivity. In addition, at the industrial scale, degree of mixing may affect the safety of a reactor system in exothermic reactions and lead to runaway reactions, which can damage equipment and cause injury to operating personnel. Hence, poorly mixed reactors can lead to low conversion, low yield, low selectivity as well as safety issues.

Generally, for efficient mixing of a reaction system, an agitator is utilized. An agitator is typically constructed of a metal shaft for strength and a metal impeller or impellers inside of the reactor and a seal mechanism to isolate the motor drive from the section that is inside the reactor. In a corrosive environment, these components may corrode, resulting in compromise of the structural integrity of the reactor and agitator and reduce the useful life of the reaction system. Therefore, there is a need to provide a method for mixing in such a corrosive environment.

When the need for efficient mixing is combined with a corrosive reaction environment, reactor design becomes doubly challenging. In certain corrosive systems, glass lined steel is commonly used to construct reactor, agitator and baffles to minimize corrosion and maintain equipment integrity. However, in systems that use HF as a reaction component, such systems are unsuitable due to the incompatibility of HF with glass.

U.S. Pat. No. 7,102,040 discloses a reactor design, which minimizes corrosion. However, U.S. Pat. No. 7,102,040 does not disclose a means for mixing the reaction contents. In fluorination systems that generate HCl (see examples below), it is typically not necessary to provide additional means of mixing because the HCl that is generated provides efficient mixing of the reactor contents as it leaves the liquid phase. Examples of such reactions are:

CCl₄+2HF→CCl₂F₂+2HCl (chemical reaction for preparation of CFC-12, CCl₂F₂)

CHCl₃+HF→CHClF₂+2HCl (chemical reaction for preparation of HCFC-22, CHClF₂)

CHCl₂CH₂CCl₃+5HF→CHF₂CH₂CF₃+5HCl (chemical reaction for preparation of HFC-245fa, CHF₂CH₂CF₃)

Regarding fluorination reactions in particular, reactors that are lined with a loose lining fabricated from fluoropolymer materials have been found to be useful for combating the corrosive conditions present in certain small-scale liquid phase fluorination reactions. For example, U.S. Pat. No. 5,902,912 teaches using a 50 gallon (appx. 6.7 ft³) loosely lined reactor vessel for producing less than one million lbs/yr of fluorocarbons in pilot scale operations. However, it has been determined that conventional non-corroding, fluoropolymer-lined reactors suffer from a variety of problems when utilized in large-volume processes, e.g. at least about 1000 gallons (appx. 134 ft³). Such problems include body flange seal leaking, liner flexing stress and shrinking, as well as leakage of hydrogen fluoride through the liner. Therefore, a need exists for non-corrosive reactors that can be used for the commercial scale production of fluorinated compounds. More particularly, there is a need for a high integrity, fluoropolymer lined metallic vessel having a heat input/output capability suitable to manufacture HFCs, such as HFC-143a, HFC-32, HFC-245fa, HFC-227ea, HFC-236fa, HFC-365mfc, HCFO-1233xf, HCFC-244bb, HFO-1234yf, etc., and to conduct other highly corrosive applications on a commercial scale.

### IV. SUMMARY OF THE DISCLOSURE

The present invention provides a non-corroding and highly reliable apparatus useful for liquid phase hydrofluorination of organic compounds as well as an inventive means of agitation of the reactor contents for a more efficient reaction resulting in higher conversion, higher yield, and better selectivity combined with more economical and safer operation.

The reactor of the present invention may also be used for other chemical processing that requires mixing as well as heating or cooling. The reactor finds particular use in the manufacture of hydrochlorofluorocarbons (HCFCs)). The reactor of the invention includes a large volume reactor vessel lined with a loose fluoropolymer liner that is highly resistive to corrosion and system or systems to promote mixing with or without the use of mechanical agitation in the reactor.

An embodiment of the present invention includes a reactor apparatus comprising a reactor assembly having a reaction chamber; a compartmentalizing apparatus including a plurality of compartments, each compartment being open at a top end and bottom end, the compartmentalizing apparatus being disposed within the reaction chamber; and an inlet disposed at an area of the reactor and in fluid communication between the reaction chamber and a feed source.

Another embodiment of the present invention includes: a reactor apparatus having a reactor assembly having a reaction chamber; at least one agitator assembly configured to generate mixing within the reaction chamber, the agitator assembly including: a shaft partially lined or coated with a fluoropolymer or other non-metallic corrosion-resistant material; an impeller lined or coated with the fluoropolymer or other non-metallic corrosion-resistant material; and an inlet disposed at an area of the reactor and in fluid communication between the reaction chamber and a feed source.

The agitator assembly also includes: a primary seal disposed at a portion of a wall of the reaction chamber and between the reaction chamber and the shaft of the agitator assembly, the primary seal being configured to prevent leakage of liquids or gases from the reaction chamber to an outside environment; and a secondary seal disposed along the shaft of the agitator assembly at a defined distance below the primary seal, the secondary seal being configured to seal between the shaft of the agitator assembly and the wall of the reaction chamber, wherein the primary seal and the secondary seal are configured to allow rotation of the shaft of the agitator assembly.

Additionally, an embodiment of the present invention includes at least one agitator assembly having: a seal cup disposed along the shaft of the agitator assembly at a position below the secondary seal; a liner of fluoropolymer or other non-metallic corrosion-resistant material extending from the wall of the reaction chamber into the seal cup; and a seal fluid disposed and held in the seal cup, the seal fluid being a fluid compatible with a reaction performed in the reaction chamber.

### V. BRIEF DESCRIPTION OF THE DRAWINGS

These and other features, aspects, and advantages of the present invention will become better understood with regard to the following description, appended claims, and accompanying drawings wherein:

FIG. 1 illustrates an embodiment of a compartmentalizing apparatus of the present invention;

FIG. 2 illustrates an embodiment of an agitator assembly of the present invention;

FIG. 3 illustrates another embodiment of the agitator assembly of the present invention.

### VI. DETAILED DESCRIPTION OF DISCLOSURE

The foregoing summary and general description of the invention and the ensuing detailed description are exemplary and explanatory and are not restrictive of the invention, as defined in the appended claims. Other features and embodiments and modifications will be apparent from the present description and are within the scope of the invention. The entire contents of U.S. Pat. Nos. 8,258,355, 8,084,653, and U.S. Published Patent Application No. 2007/0197842 are incorporated herein by reference.

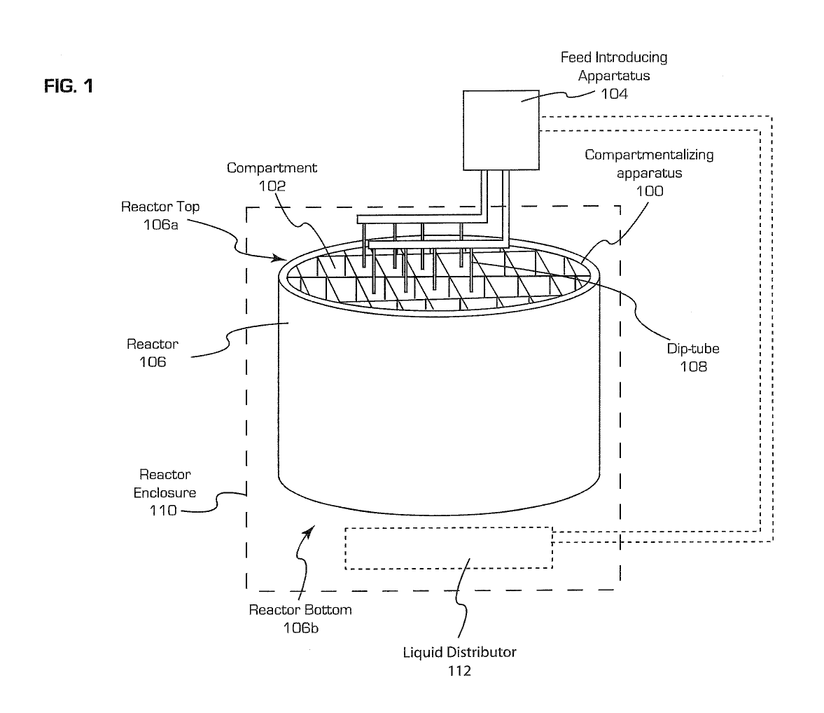

In one embodiment, the invention provides a reactor apparatus comprising a vessel inside of which a compartmentalizing apparatus 100 is placed to compartmentalize the commercial size reactor to more closely approximate a small scale reactor with a length to diameter ratio of at least 2:1 where mixing is more efficient due to smaller diameter as well as due to wall effects which increase turbulence. This type of small scale reactor can be referred to as a bubble column reactor. Referring to FIG. 1, a compartmentalizing apparatus 100 includes a plurality of compartments 102 that are open on top and bottom. The shape of each compartment 102 may be rectangular, circular, hexagonal, etc or a combination of these shapes. The compartments 102 are constructed of a material that is both corrosion resistant and efficient for heat transfer, such as silicon carbide, graphite and the like.

A feed introducing apparatus 104 introduces individual feeds to each compartment 102. Alternatively the feed introducing apparatus 104 introduces feed to multiple compartments 102 organized as a group.

The present embodiment eliminates the need for mechanical agitation. Instead each compartment behaves similar to a bubble column reactor of a much smaller size.

The compartmentalizing apparatus 100 need not be attached to the inner walls of the reactor 106 (i.e. reaction chamber). The reactor 106 is provided within a sealed reactor enclosure 110. The feeds to the reactor enter through the bottom 106b of the reactor 106, a side nozzle or through the top 106a via multiple dip-tubes 108.

Alternatively, when the reactor feed is introduced from the bottom 106b of the reactor 106, a liquid or vapor distributor 112 disposed in the bottom of the reactor 106 may optionally be used to promote initial mixing and direct the flow of the reactants into the compartments 102 of the compartmentalizing apparatus 100. The distributor 112 replaces the dip-tubes 108 in this embodiment. This arrangement promotes mixing via additional turbulence and reduces channeling.

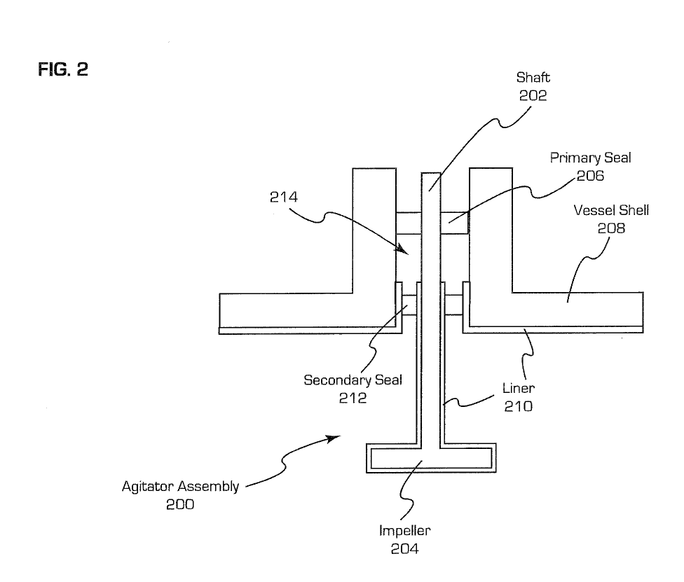

In another embodiment, a mechanical agitation assembly 200, shown in FIG. 2, is used in a reactor having a loose fluoropolymer liner 210 disposed on an inside surface of a vessel shell 208 forming the reaction chamber. The mechanical agitator assembly 200 may include a fluoropolymer lined (or coated) metal agitator shaft 202 and an impeller 204, as well as a vent for venting any HF that may permeate through the liner or coating.

A primary seal 206 is disposed at an upper end of the agitator shaft 202. The primary seal 206 acts to seal reactor gases from the environment by way of a mechanical seal, such as a double mechanical seal with a purge. Alternatively, a seal-less agitator drive, such as a magnetic drive, may be used in place of the primary seal 206. In such a seal-less embodiment, the vessel shell 208 is sealed and the agitator assembly 200 does not extend external of the vessel shell 208.

Because the reaction mixture contains HF that can permeate through the fluoropolymer liner 210, provision must be made to vent off the permeated material between the fluoropolymer liner 210 and the metal agitator shaft 202. Such provision is typically venting of the permeated material to the atmosphere outside of the reactor. Because HF is a hazardous material, the design of such a system presents a challenge to capture or to dispose the hazardous material. Moreover, a completely polymer lined agitator shaft 202 can present a very challenging mechanical seal design, since the fluoropolymer liner 210 would be in constant rotating and sealing contact.

To overcome the above problems, a novel and improved agitator design comprising a fluoropolymer lined or coated agitator shaft 202 is used. The polymer covers at least 95% of the shaft length within the vessel shell 208. To allow proper design of the agitator shaft 202 through the rotating contact and seal areas, the remaining 5% or less of the agitator shaft 202 is comprised of the same base agitator shaft metal, or of metal, metal liner, or metal coating, that is resistant to the reaction mixture vapor in the reactor vapor space. This section of 5% or less exposed metal is referred as “resistant metal”. The agitator shaft 202 need not be fabricated entirely of resistant metal. Rather, only the portion of the shaft that is not lined or coated with the fluoropolymer is constructed of resistant metal. The fluoropolymer liner 210 is typically terminated at this resistant metal. Only the polymer-lined portion of the agitator assembly 200 is immersed into liquid content.

However, because of splashing, misting, or entrainment, corrosive liquid may be present in the vapor space of the vessel. The novel agitator design includes provision to prevent contact of such liquid with the resistant metal of the shaft.

Any material such as HF permeating through the liner 210 will vent off at the point where the liner terminates at this resistant metal while still inside the vessel shell 208. To accommodate this, as well as to prevent corrosive liquid from contacting the resistant metal, a secondary seal 212 is provided on the lined section of the agitator shaft 202, below the termination of the liner. This secondary seal 212 may be a labyrinth seal, throttle bushing, or other suitable seal, as known in the industry.

A purge fluid (gas or liquid) is introduced into the space 214 between the primary seal 206 and the secondary seal 212 to actively prevent corrosive reactor mixture material from contacting the resistant metal and sweep it back into the vessel. The fluid used may be any fluid compatible with the reaction mixture and noncorrosive to the resistant metal, including reactants or products.

To promote mixing, the vessel may be equipped with baffles (not shown). These are made of metal lined or coated with fluoropolymer and may be inserted through nozzles in the top of the vessel. One to four baffles are typically employed.

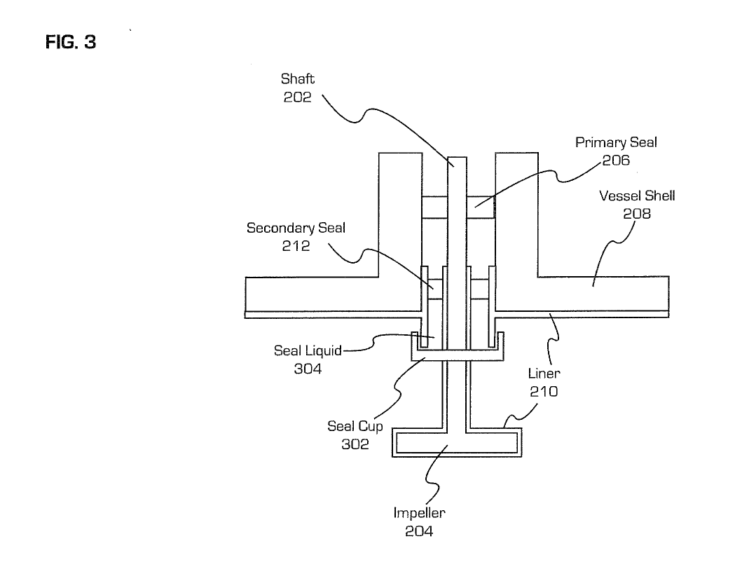

In another embodiment, as illustrated in FIG. 3, a liquid seal well cup 302 is mounted on the rotating shaft 202 at a position along the polymer-lined section 210 to form a static liquid seal to keep corrosive reactor mixture away from the metal resistant shaft. In this embodiment, the purge fluid 304 is a liquid compatible with the reaction mixture and noncorrosive to the resistant metal. The seal cup 302 can be used alone, or in combination with the secondary seal 212 as described in the previous embodiment.

The design in each embodiment allows venting of the permeated hazardous material inside the reactor vessel to be re-captured inside the reactor, while the active fluid flow covering the resistant metal portion and the agitator rotating mechanism would prevent any corrosive material from contacting the resistant metal shaft. The design will allow a conventional metal shaft and conventional primary seal designs such as a double mechanical seal or a seal-less agitator drive, to be in used in the area of constant rotation and seal contact.

The present invention also provides a hydrofluorination process using the reactor assemblies described above and shown in FIG. 1-3. In the hydrofluorination process of the present invention, a hydrochlorofluoroolefin material is supplied into the vessel through at least one inlet. Additionally, hydrogen fluoride is supplied into the vessel through at least one inlet, as well. The hydrochlorofluoroolefin is allowed to react with the hydrogen fluoride to form a hydrochlorofluoroocarbon. Depending on the particular reactor embodiment employed, the reaction is facilitated by the micro-turbulences created by the compartments 102 of the compartmentalizing apparatus 100, or by the agitation action generated by the agitator assemblies shown in FIGS. 2 and 3.

Moreover, a chlorine source may be supplied into the reaction chamber as well.

Also, a catalyst may be dosed into the reaction chamber to further promote the hydrofluorination process.

The invention further provides a process for forming hydrofluorocarbons, such as HFC-143a, HFC-32, HFC-245fa, HFC-227ea, HFC-236fa, HFC-365mfc, HCFC-244bb but not limited to these HFCs, by using the embodiments of the present invention.

In another embodiment, the present invention is a process to prepare 2-chloro-1,1,1,2-tetrafluoropropane (244bb) which comprises contacting 2-chloro-3,3,3,-trifluoropropene (1233xf) with HF in the presence of a fluorination catalyst in the reactor apparatus described herein. The fluorination catalysts contemplated in this regard are, without limitation, those known in the art, and are preferably liquid phase fluorination catalysts. A non-exhaustive list of such fluorination catalysts serviceable in the invention include: Lewis acids, transition metal halides, transition metal oxides, Group IVb metal halides, a Group Vb metal halides, or combinations thereof. Non-exclusive examples of liquid phase fluorination catalysts include antimony halide, a tin halide, a tantalum halide, a titanium halide, a niobium halide, and molybdenum halide, an iron halide, a fluorinated chrome halide, or combinations thereof. Specific non-exclusive examples of liquid phase fluorination catalysts are SbCl₅, SbCl₃, SbF₅, SnCl₄, TaCl₅, TiCl₄, NbCl₅, MoCl₆, FeCl₃, a fluorinated species of SbCl₅, a fluorinated species of SbCl₃, a fluorinated species of SnCl₄, a fluorinated species of TaCl₅, a fluorinated species of TiCl₄, a fluorinated species of NbCl₅, a fluorinated species of MoCl₆, a fluorinated species of FeCl₃, or combinations thereof. Antimony pentachloride, SbCl₅, is preferred, with a fluorinated species of SbCl₅ more preferred.

In still another embodiment, the present invention is a process to prepare compounds such as 2,3,3,3-tetrafluoropropene (1234yf). For example, the reactor apparatus of the invention may be employed in a multi-step process to make 1234yf. In a preferred embodiment in this regard, the reactor apparatus of the present invention can be employed in the second step of a three step integrated manufacturing process for making 2,3,3,3-tetrafluoropropene. The preferred starting material for this process is one or more chlorinated compounds according to Formulae I, II and/or III:

CX₂═CCl—CH₂X (Formula I)

CX₃—CCl═CH2 (Formula II)

CX₃—CHCl—CH₂X (Formula III)

wherein X is independently selected from F, Cl, Br, and I, provided that at least one X is not fluorine; Preferably, these compounds contain at least one chlorine, more preferably a majority of X is chlorine, and even more preferably all X is chlorine. Preferably, the method generally comprises at least three reaction steps.

### Step 1:

In the first step, a starting composition including one or more compounds having Formula (I), (II) or (III), preferably 1,1,2,3-tetrachloropropene (TCP or 1230xa), and/or 2,3,3,3-tetrachloropropene (also TCP or 1230xf), and/or 1,1,1,2,3-pentachloropropane (240db), reacts with anhydrous HF in a first vapor phase reactor (fluorination reactor) to produce a mixture of 2-chloro-3,3,3-trifluoropropene (1233xf) and HCl. Preferably the reaction occurs in the presence of a catalyst, such as a fluorinated chromium oxide. The reaction is conducted in a first vapor phase reactor, for example, at a reaction temperature of about 100-400° C. and a reaction pressure of about 0-200 psig. The effluent stream exiting the vapor phase reactor may optionally comprise additional components, such as un-reacted HF, underfluoinated intermediates, and HFC-245cb.

In case of a vapor phase process, the reactor is filled with a vapor phase fluorination catalyst. Any fluorination catalysts known in the art may be used in this process. Suitable catalysts include, but are not limited to chromium, aluminum, cobalt, manganese, nickel and iron oxides, hydroxides, halides, oxyhalides, inorganic salts thereof and their mixtures. Combinations of catalysts suitable for the present invention nonexclusively include Cr₂O₃, FeCl₃/C, Cr₂O₃/Al₂O₃, Cr₂O₃/AlF₃, Cr₂O₃/carbon, CoCl₂/Cr₂O₃/Al₂O₃, NiCl₂/Cr₂O₃/Al₂O₃, CoCl₂/AlF₃, NiCl₂/AlF₃ and mixtures thereof. Chromium oxide/aluminum oxide catalysts are described in U.S. Pat. No. 5,155,082 which is incorporated herein by reference. Chromium (III) oxides such as crystalline chromium oxide or amorphous chromium oxide are preferred with amorphous chromium oxide being most preferred. Chromium oxide (Cr₂O₃) is a commercially available material which may be purchased in a variety of particle sizes. Fluorination catalysts having a purity of at least 98% are preferred. The fluorination catalyst is present in an excess but in at least an amount sufficient to drive the reaction.

### Step 2:

In the second step, the reaction apparatus of the present invention is employed whereby 1233xf, produced in Step 1, is converted to 244bb. Such a process may be performed in a temperature range of about 70-120° C. and about 50-120 psig. Fluorination catalysts as described above may be used. Such catalysts can be readily regenerated by any means known in the art if they become deactivated. One suitable method of regenerating the catalyst involves flowing a stream of chlorine through the catalyst. For example, from about 0.002 to about 0.2 lb per hour of chlorine can be added to the liquid phase reaction for every pound of liquid phase fluorination catalyst. This may be done, for example, for from about 1 to about 2 hours or continuously at a temperature of from about 65° C. to about 100° C.

### Step 3:

In the third step, the 244bb, produced from Step 2 in accordance with the invention, is fed to a second vapor phase reactor (dehydrochlorination reactor) to be dehydrochlorinated to make the desired product 2,3,3,3-tetrafluoropropene (1234yf). This reactor contains a catalyst that can catalytically dehydrochlorinate 244bb to make 1234yf.

The catalysts here may be metal halides, halogenated metal oxides, neutral (or zero oxidation state) metal or metal alloy, or activated carbon in bulk or supported form.

When metal halides or metal oxides catalysts are used, preferably mono-, bi-, and tri-valent metal halides, oxide and their mixtures/combinations, and more preferably mono-, and bi-valent metal halides and their mixtures/combinations. Component metals include, but are not limited to, Cr³⁺, Fe³⁺, Mg²⁺, Ca²⁺, Ni²⁺, Zn²⁺, Pd²⁺, Li⁺, Na⁺, K⁺, and Cs⁺. Component halogens include, but are not limited to, F⁻, Cl⁻, Br⁻, and I⁻. Examples of useful mono- or bi-valent metal halide include, but are not limited to, LiF, NaF, KF, CsF, MgF₂, CaF₂, LiC1, NaCl, KCl, and CsCl. Halogenation treatments can include any of those known in the prior art, particularly those that employ HF, F₂, HCl, Cl₂, HBr, Br₂, HI, and I₂ as the halogenation source.

When neutral, i.e., zero valent, metals, metal alloys and their mixtures are used. Useful metals include, but are not limited to, Pd, Pt, Rh, Fe, Co, Ni, Cu, Mo, Cr, Mn, and combinations of the foregoing as alloys or mixtures. The catalyst may be supported or unsupported. Useful examples of metal alloys include, but are not limited to, SS 316, Monel 400, Inconel 825, Inconel 600, and Inconel 625.

Preferred catalysts include activated carbon, stainless steel (e.g. SS 316), austenitic nickel-based alloys (e.g. Inconel 625), nickel, fluorinated 10% CsCl/MgO, and 10% CsCl/MgF₂. The reaction temperature is preferably about 300-550° C. and the reaction pressure is preferably about 0-150 psig. Preferably, the reactor effluent is fed to a caustic scrubber or to a distillation column to remove the by-product of HCl to produce an acid-free organic product which, optionally, may undergo further purification.

### Example 1

A reactor apparatus is constructed as described above and having a 3,000 gallon interior volume (11,353 Liters). The reactor is pre-charged with antimony pentachloride catalyst. A fluorochlorinated organic and hydrogen fluoride are then introduced into the reactor. The operating conditions of the reactor are set to 100 psig and 230° F., and steam is introduced on the steam jacket to heat the reactor. The process yields at least 4,000 lb/hr of a hydrochlorofluorocarbon, and accumulates at least 2000 hours of operating time without leaks, liner damage or damage to the mixing system.

### Example 2

A reactor apparatus is constructed as described above and having a 3,000 gallon interior volume (11,353 Liters). The reactor is pre-charged with a predetermined amount of antimony pentachloride catalyst and HF. A fluorochlorinated organic and hydrogen fluoride are then continuously fed to the reactor. The operating conditions of the reactor are set to 100 psig and 230° F., and steam is introduced on the steam jacket to heat the reactor. Conversion of the organic is monitored and additional charges of antimony pentachloride catalyst is made batchwise and/or continuously to keep the conversion above 90%. The process yields at least 4,000 lb/hr of a hydrochlorofluorocarbon, and accumulates at least 2000 hours of operating time without leaks, liner damage or damage to the mixing system.

### Example 3

A reactor is constructed as described above and having a 2500 gallon (9462 liter) interior volume. It is equipped with a 5 HP top mounted agitator. The agitator consists of an impeller and shaft, both encapsulated with polytetrafluoroethylene (PTFE). The agitator is driven by a magnetic coupling, and a labyrinth seal is provided below the coupling. The shaft liner ends in the space between the magnetic coupling and the labyrinth seal. A purge stream of nitrogen at a rate of about 2.0 SCFM is introduced into the space between the magnetic coupling and labyrinth seal. Two PTFE lined baffles are provided. The reactor is pre-charged with a predetermined amount of antimony pentachloride catalyst and HF. A fluorochlorinated organic and hydrogen fluoride are then continuously fed to the reactor. The operating conditions of the reactor are 100 psig and 190° F. The process yields 4,000 lb/hr of a hydrochlorofluorocarbon.

What is claimed is:

1. A reactor apparatus comprising:
a reactor assembly having a reaction chamber;
a compartmentalizing apparatus comprising a plurality of compartments, each compartment being open at a top end and bottom end, the compartmentalizing apparatus being disposed within the reaction chamber; and
an inlet disposed at an area of the reactor and in fluid communication between the reaction chamber and a feed source.

2. The apparatus as in claim 1, wherein the inlet comprises a plurality of dip-tubes, each dip-tube being associated with a corresponding compartment of the compartmentalizing apparatus.

3. The apparatus as in claim 1, further comprising a liquid or vapor distributor disposed at a bottom portion of the reactor, the distributor configured to: promote initial mixing, direct a flow of reactants into the plurality of compartments, induce mixing up to and including turbulence, and reduce channeling.

4. An apparatus comprising:
a reactor assembly having a reaction chamber;
at least one agitator assembly configured to generate mixing within the reaction chamber, the agitator assembly comprising:
a shaft partially or fully lined or coated with a fluoropolymer or other non-metallic corrosion-resistant material, and
an impeller lined or coated with a fluoropolymer or other non-metallic corrosion-resistant material; and
an inlet disposed at an area of the reactor and in fluid communication between the reaction chamber and a feed source.

5. The apparatus as in claim 4, wherein the at least one agitator assembly is magnetically driven.

6. The apparatus as in claim 4, wherein the at least one agitator assembly further comprises:
a primary seal disposed at a portion of a wall of the reaction chamber and between the reaction chamber and the shaft of the agitator assembly, the primary seal being configured to prevent leakage of liquids or gases from the reaction chamber to an outside environment; and
a secondary seal disposed along the shaft of the agitator assembly at a defined distance below the primary seal, the secondary seal being configured to seal between the shaft of the agitator assembly and the wall of the reaction chamber,
wherein the primary seal and the secondary seal are configured to allow rotation of the shaft of the agitator assembly.

7. The apparatus as in claim 6, wherein the primary seal is positioned at a region of the shaft of the agitator assembly bare of the fluoropolymer or other non-metallic corrosion-resistant material, and the secondary seal is positioned at a region of the shaft of the agitator assembly lined or coated with the fluoropolymer or other non-metallic corrosion-resistant material.

8. The apparatus as in claim 6, wherein a volume between the primary seal and the secondary seal contains a purge fluid, the purge fluid being compatible with a reaction performed in the reaction chamber.

9. The apparatus as in claim 7, wherein the secondary seal is formed of a labyrinth sleeve.

10. The apparatus as in claim 7, wherein the secondary seal is formed of a throttle bushing.

11. The apparatus as in claim 6, wherein the at least one agitator assembly further comprises:
a seal cup disposed along the shaft of the agitator assembly at a position below the secondary seal;
a fluoropolymer or other non-metallic corrosion-resistant liner material extending from the wall of the reaction chamber into the seal cup; and
a seal fluid disposed and held in the seal cup, the seal fluid being a fluid compatible with a reaction performed in the reaction chamber.

12. A hydrofluorination method comprising:
providing a reactor apparatus having a compartmentalizing apparatus disposed therein;
supplying a hydrochlorofluoroolefin material into compartments of the compartmentalizing apparatus through at least one inlet;
supplying hydrogen fluoride into the compartments through the at least one inlet;
reacting the hydrochlorofluoroolefin with the hydrogen fluoride to form a hydrofluorochlorocarbon.

13. The method as in claim 12, further comprising supplying a chlorine source into the compartments through the at least one inlet.

14. The method as in claim 12, further comprising supplying doses of catalyst into the compartments through the at least one inlet.

15. A hydrofluorination method comprising:
providing a reactor apparatus having an agitator assembly at least partially lined or coated with a fluoropolymer or other non-metallic corrosion-resistant material disposed within a reaction chamber of the reactor apparatus;
supplying a hydrochlorofluoroolefin material into the reaction chamber through at least one inlet;
supplying hydrogen fluoride into the reaction chamber through the at least one inlet;
reacting the hydrochlorofluoroolefin with the hydrogen fluoride to form a hydrofluorochlorocarbon.

16. The method as in claim 15, further comprising supplying chlorine into the reaction chamber through the at least one inlet.

17. The method as in claim 15, further comprising supplying doses of catalyst into the reaction chamber through the at least one inlet.

18. A process to prepare 2-chloro-1,1,1,2-tetrafluoropropane (244bb) comprising contacting 2-chloro-3,3,3,-trifluoropropene (1233xf) with HF in the presence of a fluorination catalyst in the reaction apparatus of claim 1 under conditions effective to produce 244bb.

19. The process of claim 18, wherein the fluorination catalyst is selected from the group consisting of SbCl₅, SbCl₃, SbF₅, SnCl₄, TaCl₅, TiCl₄, NbCl₅, MoCl₆, FeCl₃, a fluorinated species of SbCl₅, a fluorinated species of SbCl₃, a fluorinated species of SnCl₄, a fluorinated species of TaCl₅, a fluorinated species of TiCl₄, a fluorinated species of NbCl₅, a fluorinated species of MoCl₆, a fluorinated species of FeCl₃, or combinations thereof.

20. A process to prepare 2,3,3,3-tetrafluoropropene (1234yf) comprising:
a) providing a starting composition comprising at least one compound having a structure selected from Formula I, II and II:
CX₂═CCl—CH₂X (Formula I)
CX₃—CCl═CH₂ (Formula II)
CX₃—CHCl—CH₂X (Formula III)
wherein X is independently selected from F, Cl, Br and I, provided that at least one of X is not F;
b) contacting said starting composition with HF under conditions effective to produce a first intermediate composition comprising 2-chloro-3,3,3-trifluoropropene (1233xf);
c) contacting said first intermediate composition comprising 1233xf with HF in the presence of a fluorination catalyst in the reaction apparatus of claim 1 under conditions effective to produce a second intermediate composition comprising 244bb; and
d) dehydrochlorinating at least a portion of said 244bb to produce a reaction product comprising 1234yf.

21. A process to prepare 2-chloro-1,1,1,2-tetrafluoropropane (244bb) comprising contacting 2-chloro-3,3,3,-trifluoropropene (1233xf) with HF in the presence of a fluorination catalyst in the reaction apparatus of claim 3 under conditions effective to produce 244bb.

22. The process of claim 21, wherein the fluorination catalyst is selected from the group consisting of SbCl₅, SbCl₃, SbF₅, SnCl₄, TaCl₅, TiCl₄, NbCl₅, MoCl₆, FeCl₃, a fluorinated species of SbCl₅, a fluorinated species of SbCl₃, a fluorinated species of SnCl₄, a fluorinated species of TaCl₅, a fluorinated species of TiCl₄, a fluorinated species of NbCl₅, a fluorinated species of MoCl₆, a fluorinated species of FeCl₃, or combinations thereof.

23. A process to prepare 2,3,3,3-tetrafluoropropene (1234yf) comprising:
a) providing a starting composition comprising at least one compound having a structure selected from Formula I, II and II:
CX₂═CCl—CH₂X (Formula I)
CX₃—CCl═CH₂ (Formula II)
CX₃—CHCl—CH₂X (Formula III)
wherein X is independently selected from F, Cl, Br and I, provided that at least one of X is not F;
b) contacting said starting composition with HF under conditions effective to produce a first intermediate composition comprising 2-chloro-3,3,3-trifluoropropene (1233xf);
c) contacting said first intermediate composition comprising 1233xf with HF in the presence of a fluorination catalyst in the reaction apparatus of claim 3 under conditions effective to produce a second intermediate composition comprising 244bb; and
d) dehydrochlorinating at least a portion of said 244bb to produce a reaction product comprising 1234yf.
